(19) United States
(12) Patent Application Publication
(10) Pub. No.: US 2023/0190124 A1
(43) Pub. Date: Jun. 22, 2023

(54) BODY-WORN DEVICE FOR MEASURING BLOOD FLOW

(71) Applicants: BAXTER INTERNATIONAL INC., Deerfield, IL (US); BAXTER HEALTHCARE SA, Glattpark (Opfikon) (CH)

(72) Inventors: James Patrick McCanna, Pleasanton, CA (US); Erik Edwin Tang, San Diego, CA (US); Marshal Singh Dhillon, San Diego, CA (US); Matthew John Banet, San Diego, CA (US); Sung Kwon, St. Paul, MN (US); Daniel McPeak, Minneapolis, MN (US); Jim Studer, Beldenville, WI (US)

(21) Appl. No.: 18/082,957

(22) Filed: Dec. 16, 2022

Related U.S. Application Data
(60) Provisional application No. 63/291,653, filed on Dec. 20, 2021.

(51) Int. Cl. A61B 5/026; A61B 5/00

(52) U.S. Cl. CPC A61B 5/0261; A61B 5/0022; A61B 5/6833; A61B 2562/0219

(57) ABSTRACT

Disclosed herein are systems, devices, and methods for measuring blood flow.

### CROSS-REFERENCE TO RELATED APPLICATION

The present application claims the benefit of U.S. Provisional Application No. 63/291,653, filed Dec. 20, 2021, the entire contents of which is incorporated by reference herein.

### FIELD

The present Specification relates to systems for monitoring perfusion of vessels (e.g. arteries and veins) in patients, for example after surgery, in both hospital and home environments.

### BACKGROUND

Measurement of blood flow is of critical importance, for example in the post-surgical tissue environment. Current systems typically measure post-surgical blood flow with Doppler-based technologies that can contact a patient but are not worn. Such systems do not facilitate continuous measurement. While the prior art describes some body-worn systems that attach directly to an underlying vessel and measure blood flow, these are typically based on capacitance sensors, which can be prone to lower signal-to-noise ratios.

### SUMMARY

The instant disclosure provides a novel class of blood-flow measurement systems, devices, and methods that utilize impedance signals, rather than capacitance. The instantly disclosed systems and devices have higher signal-to-noise ratios compared to related techniques previously known in the art, thereby enabling more accurate measurement of arterial and venous blood flow in underlying vessels.

For example, in embodiments, disclosed flow sensor systems can comprise;
a. an impedance system comprising at least one current-injecting electrode configured to inject an electrical current into the vessel, and at least one signal-measuring electrode configured to measure an impedance signal affected by the injected electrical current and blood flow in the vessel, wherein the current-injecting and signal-measuring electrodes are connected directly to the vessel; and
b. a processing system configured to receive the impedance signal from the impedance system, or a signal determined therefrom, and then process it to determine a parameter related to the blood flow in the vessel.

Further disclosed embodiments comprise;
a. an electrical system comprising at least one current-injecting electrode configured to inject an electrical current into the vessel, and at least one signal-measuring electrode configured to measure an electrical signal affected by the injected electrical current and blood flow in the vessel, wherein the current-injecting and signal-measuring electrodes are connected directly to the vessel; and
b. a processing system configured to receive the electrical signal from the electrical system, or a signal determined therefrom, and then process it to determine a parameter related to the blood flow in the vessel.

Further disclosed embodiments comprise;
a. an impedance system comprising at least one current-injecting electrode configured to inject an electrical current into the vessel, and at least one signal-measuring electrode configured to measure an impedance signal affected by the injected electrical current and blood flow in the vessel, wherein the current-injecting and signal-measuring electrodes are connected directly to the vessel;
b. an optical system comprising a light source and a photodetector, with the light source configured to irradiate the vessel with optical radiation and the photodetector configured to detect the optical radiation after it irradiates the vessel and generate an optical signal; and
c. a processing system configured to receive the impedance signal from the impedance system and the optical signal from the optical system, or a signals determined therefrom, and collectively process them to determine a parameter related to the blood flow in the vessel.

Further disclosed embodiments comprise;
a. a body-worn patch comprising an impedance circuit;
b. an electrode system attached directly to the vessel and electrically connected to the impedance circuit; and
c. a processing system configured to receive signals from the impedance circuit and process them to estimate blood flow in the vessel.

Disclosed embodiments can further comprise a transmitter.

Disclosed embodiments can further comprise a housing.

Disclosed embodiments also comprise kits comprising the disclosed systems and devices.

Disclosed embodiments also comprise methods of use of the disclosed systems and devices.

### BRIEF DESCRIPTION OF THE DRAWINGS

FIG. 1 is a photograph of a patient wearing a patch for monitoring perfusion of underlying vessels according to the disclosure.

FIG. 2 is a schematic drawing of an impedance system used in the patch of FIG. 1 to measure perfusion of a patient’s vein.

FIG. 3 is a schematic drawing showing the impedance system of FIG. 2 making an impedance measurement from a vein to determine perfusion.

FIG. 4 is a graph showing impedance values measured at different frequencies using the impedance system of FIG. 3.

FIG. 5 is a schematic drawing showing the impedance system of FIG. 2 making a capacitance measurement from a vein to determine perfusion.

FIG. 6 is a graph showing capacitance values measured at different frequencies using the impedance system of FIG. 5.

FIG. 7 is a schematic drawing showing current modulated at both low and high frequencies that, respectively, passes around and through blood cells disposed in an underlying vessel.

FIG. 8 is a schematic drawing of a near infrared spectroscopy system used in an alternate embodiment of the patch of FIG. 1 to measure perfusion from a vein.

FIG. 9 is a graph of a time-dependent waveform measured with the impedance system of FIG. 2 from a ‘phantom’ of a human vessel during periods when synthetic blood is not flowing and flowing through the vessel.

FIGS. 10A and 10B are, respectively, an image and a photograph of a circuit board used in the patch according to the disclosure.

FIG. 11 is a schematic drawing of an impedance circuit deployed in the circuit board of FIGS. 10A and 10B.

FIG. 12A is a schematic drawing of an electrode patch used to measure perfusion from a patient according to a further embodiment of the disclosure.

FIG. 12B is a schematic drawing of a bottom surface of a circuit board that connects to the electrode patch shown in FIG. 12A to measure perfusion from the patient.

FIG. 12C is a schematic drawing of an enclosure that encloses the circuit board of FIG. 12B.

FIG. 13 shows a disclosed embodiment comprising a patient, a wireless gateway, a cloud-based system, and data analytics.

### DETAILED DESCRIPTION

Disclosed embodiments comprise wearable blood flow measurement systems and devices.

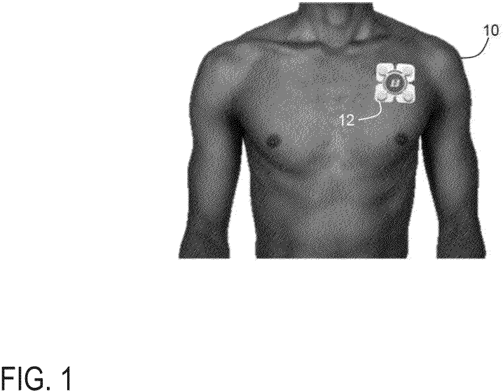

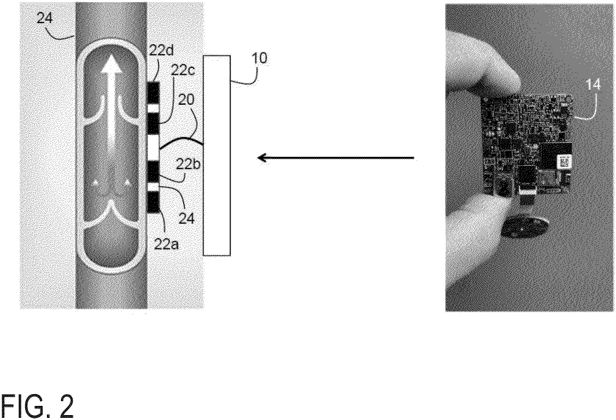

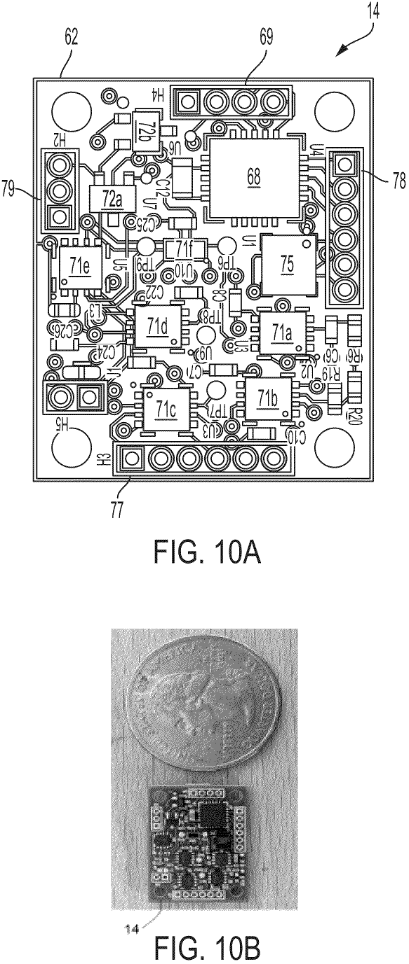

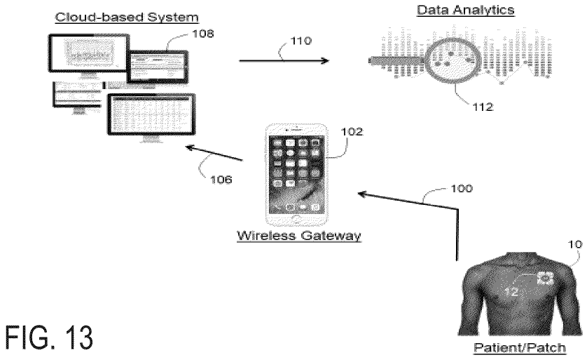

As shown in FIG. 1, a disclosed embodiment comprises a wearable “patch” 12 worn by a patient 10 that monitors blood flow in an underlying vessel, e.g. a vein or artery, using a collection of sensors described in more detail below. In embodiments, the sensors can connect directly to the vessel or be disposed above tissue that surrounds the vessel. In embodiments, the patch 12 features an electronics unit 18 that encloses a circuit board (as shown in more detail in FIGS. 2, 10A, and 10B) that controls functions of the patch 12, e.g. operation of the sensors to measure physiological signals from the patient 10, analysis of the physiological signals to determine information related to blood flow in the underlying vessels, and wireless transmission of the information to external systems for follow-on evaluations (as shown in more detail in FIG. 13). In embodiments, an adhesive component 11, e.g. an adhesive foam backing supporting multiple hydrogel electrodes, secures the patch 12 to the patient 10 so that it can be continuously worn for a period of time, e.g. during a hospital stay or for a few weeks afterwards. For example, in embodiments, disclosed devices can be worn for the duration of a hospital stay. In further embodiments, the device can be worn for 1 or more weeks post-discharge, or more. In embodiments the device can be worn intermittently.

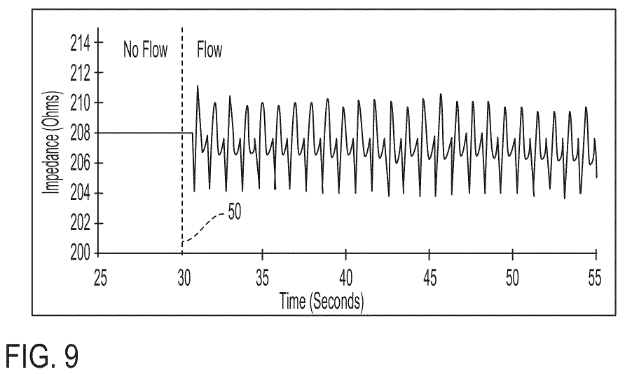

In embodiments, a clinician applies the patch 12 to the patient 10 after a surgery, e.g. a reconstructive surgery involving a skin flap (such as breast reconstructive surgery). When applied to tissue such as a skin flap, sensors in the patch 12 monitor blood flow (i.e. perfusion) in the underlying tissue, and thereby indicate if the flap is well-perfused and the patient is effectively recovering post-surgery. In embodiments, good perfusion indicates a successful recovery; in contrast, poor perfusion indicates the skin flap is not receiving adequate amounts of blood and is in danger of becoming necrotic. For example, in embodiments, “good” perfusion values are characterized by a signal featuring a quasi-periodic pulsatile component that occurs at a frequency matching the patient’s “true” heart rate, as measured with more conventional means (e.g. an electrocardiogram). See, for example, the signal after about 30 seconds in FIG. 9. The term “matching”, as used in this context, means the heart rate measured from the signal is within 15% of the patient’s true heart rate, which is typically between 30 and 200 beat/min. Such signals indicate heartbeat-induced blood is adequately flowing through the vessel. In contrast, “poor” perfusion values are characterized by a signal that completely lacks a pulsatile component (see, for example, the signal before about 30 seconds in FIG. 9), or features a sporadic, non-periodic pulsatile component or quasi-periodic pulsatile component that does not match the patient’s true heart rate.

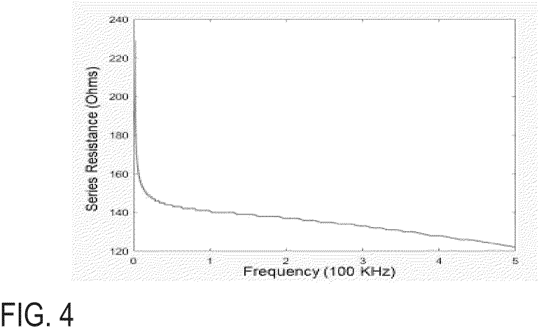

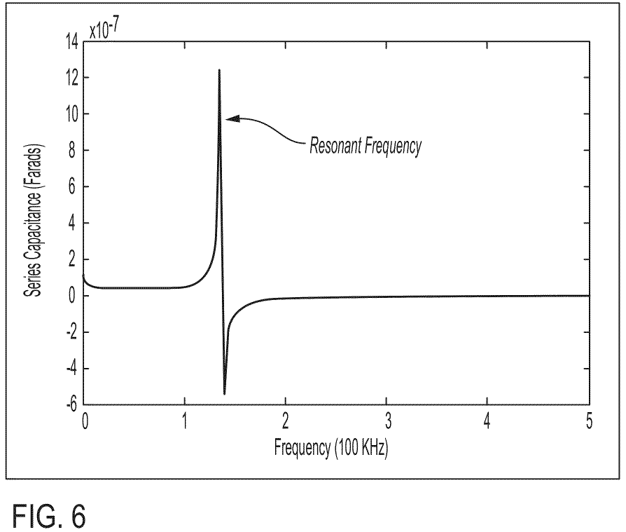

FIGS. 2-6 show in more detail how the disclosed patch 12 measures physiological signals related to blood flow in an underlying vessel, which in this case is a vein 25. In the embodiment shown in FIG. 2, the patch 12 adheres to a skin surface 27 using the adhesive component 11. The electronics unit 18 within the patch 12 connects through a thin 4-wire cable 20 made of resorbable materials to an electrode system 24 that attaches directly to an outer surface of the underlying vein 25. The electrode system 24 performs a ‘complex’ bioimpedance measurement, which is typically modeled by a mathematical equation featuring both real and imaginary components, as described with reference to FIGS. 4 and 6. Such a mathematical equation ultimately determines the time-dependent resistance and capacitance of the vein 25 at the area of measurement. Further analyses of these parameters indicate the blood flow therein.

In embodiments, the resorbable materials within the 4-wire cable 20 primarily comprise conductive materials that can safely dissolve within the patient’s body without causing any adverse reaction. Such materials, for example, can comprise conductive polymers and benign metals such as magnesium, manganese, iron and zinc, or combinations (e.g. alloys) thereof. In embodiments, alloys that contain these metals and other additives, such as RESOLOY^{®} (https://www.meko.de/innovations/resoloy), a conductive, resorbable material commonly used in stents, may also be used.

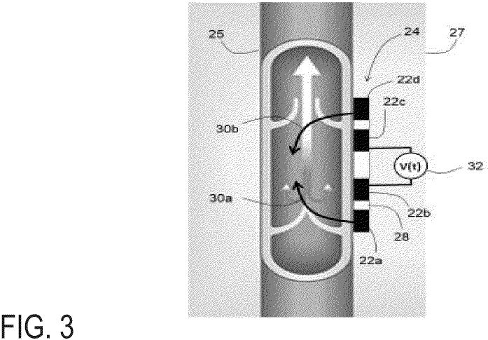

In embodiments, the electrode system 24 features four distinct electrodes 22a-d separated by an insulating and resorbable polymeric material 28 that prevents the electrodes 22a-d from contacting each other. In embodiments, within the electrode system 24 are pairs of “sense” electrodes 22b,c and ‘drive’ electrodes 22a,d, with the sense electrodes 22b,c typically located within distal drive electrodes 22a,d. In embodiments, during a measurement, the drive electrodes 22a,d inject a high-frequency, low-amperage current into the vessel. In disclosed embodiments the current’s frequency can range from, for example, 5-500 kHz, and its mean AC amperage can range from, for example, 0.1-1.0 mA. In embodiments, current injected from one drive electrode 22a is typically out of phase, for example 90° out of phase, with current injected from the opposing drive electrode 22d. In embodiments, the circuit board 14 within the electronics unit 18 comprises an impedance circuit that sequentially performs measurements of resistance (the “real” component of bioimpedance) and reactance (the “imaginary” component of bioimpedance), as shown in more detail in FIGS. 3-6.

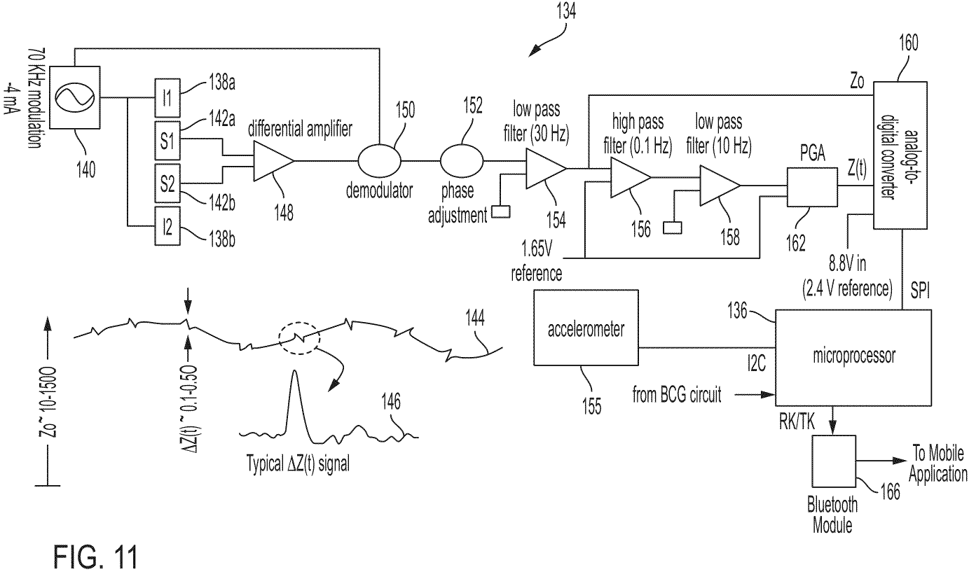

FIG. 10A shows the circuit board 14 and its electrical elements in more detail. In embodiments, the impedance circuit comprises a collection of discrete electrical components, e.g. operational amplifiers, resistors, and capacitors, as indicated in more detail in FIG. 11. In further embodiments the impedance circuit is integrated into a single small-scale semiconductor analog front end or ‘chip’, such as the MAX30009 manufactured by Maxim Semiconductor (Sunnyvale, CA). A battery 19 typically powers the circuit board 14, for example a rechargeable lithium-ion battery.

As shown in Eq. 1 below, the impedance of a system is typically represented by as a complex quantity ‘Z’, with the polar form of Z including both its magnitude and phase characteristics:

$$Z = \left|Z\right| {e}^{l a r g \left(Z\right)}$$

where the magnitude |Z| represents the ratio of the voltage amplitude to the current amplitude, and the argument arg(Z) is the phase difference between the voltage and current. Eq. 1 can be represented in Cartesian form as:

$$Z = R + I X$$

where the real part of the impedance is the resistance R and the imaginary part is the reactance X.

The reactance of the system includes contributions from both its capacitance and inductance. For a biological system such as a vessel (e.g. either an artery or a vein), the inductance of the system is small and effectively non-existent, meaning the reactance of the system effectively indicates the system’s capacitance.

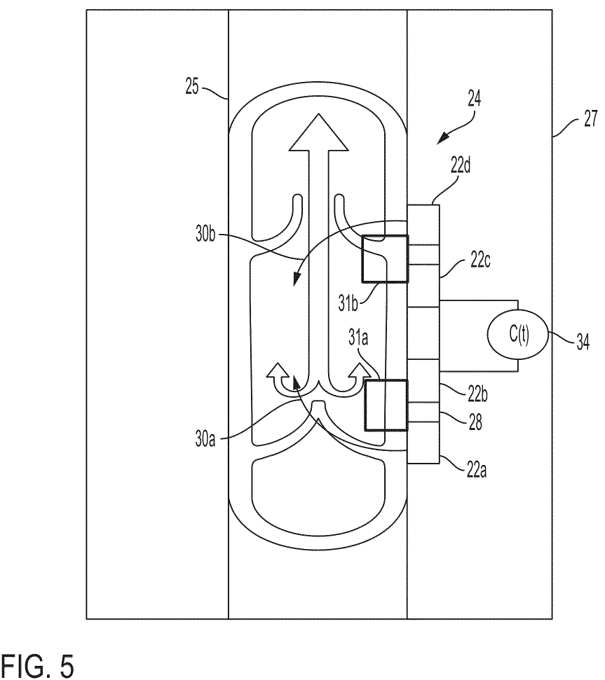

FIGS. 3 and 5 show the impedance measurement as applied to human tissue (e.g. a skin flap containing a vein and/or artery) in more detail. As indicated above, during a measurement, distal drive electrodes 22a,d inject high-frequency, low-amperage current into the vein 25, as indicated, respectively, by arrows 30a,b. If the vein 25 (or alternatively artery) is well-perfused, blood flows through it in a pulsatile manner dictated by the patient’s cardiac properties, e.g. their heart rate (herein “HR”, typically expressed in beats/minute), stroke volume (herein “SV”, typically expressed in units of volume/beat, e.g. mL), and cardiac output (the product of HR and SV, typically expressed in liters/minute). More specifically, blood is an effective electrical conductor, primarily because of iron-containing hemoglobin proteins within the red blood cells. Thus, flowing blood modulates (and typically lowers) the resistance and reactance of the vein 25 in a time-dependent manner similar to that shown in FIG. 9, i.e. a “waveform” featuring a collection of pulses occurring at a rate and magnitude dictated, respectively, by the patient’s HR and SV.

In embodiments, during a measurement, a microprocessor within the circuit board operating an algorithm 14 analyzes pulses measured by the impedance circuit to determine if blood is flowing through the vessel (indicating good perfusion) or not (indicating bad perfusion). In embodiments, the impedance circuit generates a time-dependent analog voltage measured by the sense electrodes 22b,c, as indicated by “V(t)” shown in component 32. In embodiments, an analog-to-digital converter coupled to the impedance circuit digitizes the time-dependent analog voltage to generate a digital voltage that the microprocessor then analyzes. More specifically, in embodiments, algorithms operating on the microprocessor (typically called “beatpicking” algorithms) analyze digitized time-dependent waveforms generated by the impedance circuit to determine if blood is flowing in the vein 25.

The data shown in FIG. 9 represents V(t), e.g. a digitized time-dependent waveform. It was generated using a benchtop “phantom” representing a surgical skin flap featuring a synthetic vein embedded in agar (representing surrounding connective tissue), and peristaltic pump that pumps a viscoelastic fluid with mechanical and electrical properties matched to human blood. A synthetic skin layer capped the agar and synthetic vein, and a patch featuring an impedance circuit was attached to the skin. A 4-wire cable connected the impedance circuit to an electrode system with discrete sense and drive electrodes that was sutured to the synthetic vein. The impedance circuit measured the time-dependent waveform shown in the Figure. Before about 30 seconds, the peristaltic pump was turned off, meaning no viscoelastic fluid was flowing through the vein; this simulates a non-perfused vessel, and results in a static impedance and a time-dependent waveform that lacks any pulsatile component. At around 30 seconds-as indicated in the Figure by a dashed line 50—the pump is turned on, and a pulsatile component is clearly present in the time-dependent waveform. A beatpicking algorithm operating on the microprocessor analyzed these different components of the waveform to determine periods of ‘no flow’ and ‘flow’ in the benchtop phantom.

As indicated by FIG. 4, in embodiments, the resistance measured in the vessel will depend on the frequency of current injected by the drive electrodes, with the resistance gradually increasing at low frequencies and asymptotically approaching an infinite value when sampled with DC current. As described in more detail below, further embodiments comprise impedance measurements sequentially made at multiple frequencies (called “impedance spectroscopy”) to determine additional properties of the vessel that can also indicate perfusion in the tissue that contains it.

In embodiments, sense electrodes 22b,c can also be used to detect bio-electric signals that once processed yield conventional electrocardiogram waveforms (herein “ECG”). In embodiments, algorithms operating on the patch’s microprocessor can further analyze the ECG waveforms to determine the patient’s HR and more advanced parameters, such as cardiac arrythmias. Typically ECG waveforms are most effectively measured from the patient’s abdomen, for example the chest. Additionally, and as described above, the ECG waveforms can yield the patient’s true HR. Such values can then be compared to HR values measured by the impedance circuit from the underlying vessel, with a value within 15% of the true HR indicating good perfusion.

FIG. 5 shows another embodiment which is designed to measure the time-dependent reactance of the vein 25, described above as effectively the time-dependent capacitance (indicated in the Figure as “C(t)” in component 34) because the vein’s inductance is small and essentially non-existent. In an embodiment, to simplify the measurement system, the electrode system 24 is designed as a 2-terminal system with sense 22a and drive 22b electrodes on one side of the system 24 shorted together (as indicated by component 31a), and sense 22c and drive 22d electrode on an opposing distal side of the system also shorted together (as indicated by component 31b). Similar to that shown in FIG. 3, the shorted sense 22a and drive 22b electrodes on one distal end of the electrode system 24 inject high-amperage, low-frequency current at one phase into the vein 25 (as indicated by arrow 30a), while simultaneously the shorted sense 22c and drive 22d electrodes on the opposing distal end of the electrode system 24 inject high-amperage, low-frequency current (as indicated by arrow 30b) that is 90° out of phase with the first injected current. From such a measurement the time-dependent reactance (and hence capacitance C(t)) of the vessel is measured as described above. Note that while FIG. 5 shows pairs of sense and drive electrodes shorted together, a conventional 4-electrode configuration similar to that shown in FIG. 3 can also be used in this application.

For well-perfused tissue, C(t) measured from the vein is typically represented by a time-dependent waveform featuring blood flow-induced pulses similar to that shown in FIG. 9. Additionally, like the impedance measurements described herein, in embodiments the frequency of injected current in the capacitance measurement shown in FIG. 5 can be “swept”, yielding the frequency-dependent series capacitance of the vein, as indicated in FIG. 6. Such a measurement has the advantage of indicating the “resonant frequency” of the vein, which is the frequency at which the series inductance of the capacitor (albeit small for a vein) is equal but opposite to its capacitance. In an actual measurement with, for example, a skin flap, the resonant frequency of a vein therein will depend strongly on whether blood is flowing within it. Thus the microprocessor can operate an algorithm that detects the resonant frequency. Much like the pulsatile nature of the time-dependent waveform measured from it (i.e. either V(t) and/or C(t)), an algorithm can analyze the value of the resonant frequency to determine if the vessel is perfused or not.

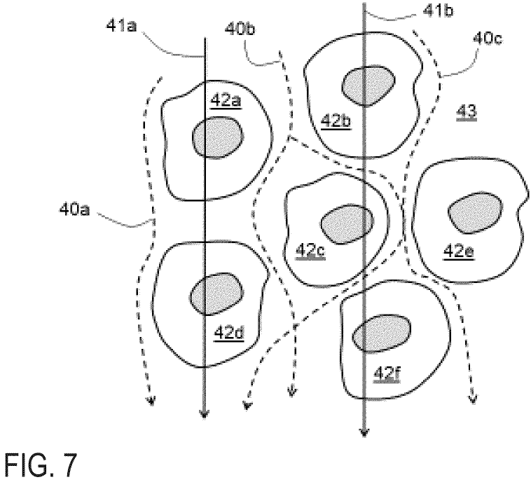

In related embodiments, disclosed systems can make multi-frequency measurements of impedance to determine not only the presence of red blood cells in the vessel, but also to estimate the concentration of blood cells therein. For example, FIG. 7 shows a graphical representation of this phenomena that includes a series of red blood cells 42a-f immersed in a fluid medium 43 representing the blood’s serum. Both the cells 42a-f and fluid medium 43 impact the impedance measurement made by the patch described above. However, as indicated by dashed lines 40a-c, relatively low frequencies of injected current pass around the cells 42a-f. Here, the capacitance of the cells’ membranes prohibits current from passing through them. This indicates relatively low frequencies of injected current are likely to sample, and thus be more sensitive to, the extra-cellular fluids in the vein. Conversely, in embodiments, relatively high frequencies of injected current, as indicated by solid lines 41a-b, can overcome the inherent capacitance of blood cells’ membrane, and pass through both the cells 42a-f and fluid medium 42. In this way, the impedance measurement describe herein can also determine a quasi-continuous, relative concentration of blood volume therein. In embodiments, such a measurement can be coupled with an algorithm operated by the microprocessor to determine, for example, partial perfusion in the vein.

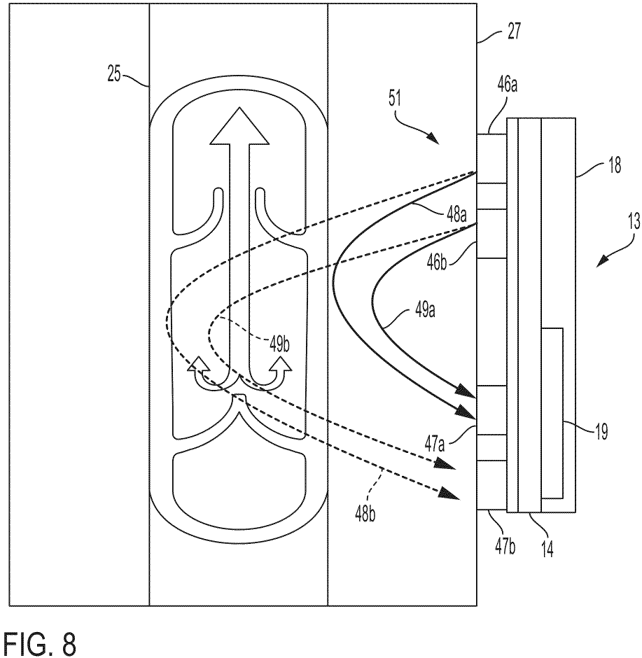

FIG. 8 shows another embodiment, featuring an alternative version of the patch 13 that includes an optical sensor 51 making a measurement of near-infrared spectroscopy (herein “NIRS”). In this embodiment the NIRS measurement may be used as a replacement for the impedance measurement described above to measure perfusion in an area of tissue, e.g., a skin flap; more preferably, the patch combines both impedance and NIRS as complementary measurements. As disclosed herein, the NIRS measurement uses sensors disposed above the vein, wherein the impedance measurement uses sensors sutured directly to the vein. In this way NIRS samples multiple vessels disposed in a relatively large area of the skin flap, as opposed to a single, specific vessel sampled by the impedance measurement. Thus, NIRS may be more effective at determining an overall perfusion of the skin flap as opposed to perfusion in a particular vessel.

In embodiments, NIRS, in general, can be effective at determining perfusion at different depths in blood-containing vessels, as they include hemoglobin, myoglobin, and other compounds that exhibit variable absorption of near-infrared light in response to changes in oxygen availability. Thus, analysis of NIRS signals can yield two parameters of the underlying tissue:
a. blood flow at different depths; and
b. changes in tissue oxygenation and regional blood volume.

During NIRS, blood propagating in a pulsatile manner in the vessel will absorb a portion of the incident infrared radiation according to Beer’s Law; the degree of absorption will depend on the cross-sectional width of the vessel and the degree of oxygenation of the light-absorbing blood therein. A perfused vessel will yield a corresponding pulsatile waveform (similar to that shown in FIG. 9) as measured by one or more photodetectors that are displaced from the source of the near-infrared radiation. In embodiments, analysis of NIRS signals measured with different optical wavelengths yields the presence of perfused vessels at different depths in the underlying tissue, as well as tissue oxygenation and the regional oxygenated total hemoglobin ratio (herein “rSO₂”) for the combined arterial, capillary, and venous hemoglobin sources underlying a given sensor. Anatomically, at any given time, the blood contained within an individual tissue segment exists in a generally accepted vascular distribution of approximately 20% arterial, 75% venous, and 5% capillary. Therefore, as NIRS provides predominantly non-capillary tissue oxygenation information, it may be considered a surrogate estimate of local tissue perfusion and oxygen utilization.

Referring again to FIG. 8, the patch 13 for making a NIRS measurement is typically disposed on the surface 27 of a tissue area such as a skin flap containing an underlying vessel, and in this case a vein 25. The optical system 51 within the patch 13 includes a first light source 46a (e.g. a light-emitting diode, herein “LED”) that emits optical radiation at a first infrared frequency (e.g. □□ = 700 nm), and a second light source 46b that emits optical radiation at a second infrared frequency (e.g. □ = 950 nm). In embodiments, radiation emitted by both light sources 46a,b propagates through the skin flap with banana-shaped profiles, with some radiation penetrating relatively shallow depths as indicated by solid curved arrows 48a, 49a, and other radiation penetrating relatively deeper depths in the tissue as indicated by dashed curved arrows 48b, 49b. Factors such as optical absorption and scattering off underlying tissue (such as the skin 27, vein 25, and other connective interstitial tissue) determines the depth of penetration. While FIG. 8 shows, respectively, curved arrows 48a,b and 49a,b indicating light from the first 46a and second 46b light sources irradiating one particular vein 25, in reality the light sources 46a,b will irradiate a collection of veins and arteries in the underlying tissue, thus providing a high-level overview of perfusion in the skin flap. In embodiments, a pair of laterally spaced photodetectors 47a,b detect radiation from the first 46a and second 46b light sources, with the most proximal photodetector 47a detecting relatively shallow-penetrating radiation (as indicated by solid curved arrows 48a, 49a), and the most distal photodetector 47b detecting relatively deep-penetrating radiation (as indicated by dashed curved arrows 48b, 49b). Typically the photodetectors 47a,b are separated from the light sources 46a,b by about 20-40 mm, such as 20 mm, 25 mm, 30 mm, 35 mm, or 40 mm; a larger separation allows detection of deeper penetrating radiation, and thus vessels that are disposed deeper in the tissue. A separation of 20-40 mm typically allows sampling of a penetration depth of about 0.33-0.5 of the separation, e.g. around 1-3 cm such as, for example, 1 cm, 2 cm, or 3 cm.

In embodiments, circuitry within the patch’s electronics module 18 controls activation of the light sources 46a,b and photodetectors 47a,b. This, in turn, yields a time-dependent analog voltage from each photodetector corresponding to a specific infrared wavelength. As with the impedance measurement described above, an analog-to-digital converter in the electronics module 18 receives and digitizes the time-dependent analog voltages from the photodetectors 47a,b to generate a set of time-dependent optical waveforms (typically called a “photoplethysmogram;” herein “PPG”) corresponding to each light source 46a,b and each photodetector 47a,b. Such waveforms, in cases wherein the tissue they originate is perfused, will have a similar shape to the pulsatile impedance waveforms shown in FIG. 9. As with this signal, the presence of a pulsatile component in the PPG indicates blood flow in the underlying tissue; conversely, lack of a pulsatile component in the PPG indicates a poorly perfused tissue.

In further embodiments, an algorithm operating on the microprocessor can compare ratios of the magnitude of the pulsatile signals to determine the relative oxygenation of blood in the underlying tissue. Such an algorithm is similar to that used in a conventional pulse oximeter, which typically features a single photodetector and lights sources centered in the red (e.g. □□ ~ 650 nm) and infrared (e.g. □ ~ 900 nm) spectral regions. In NIRS, using techniques known in the art that rely on well-known absorption spectra, the ratio of pulsatile signals can be compared to determine hemoglobin (herein “Hb”), oxygenated hemoglobin (herein “HbO₂”), and water (herein “H₂O”). As described above, these parameters, taken alone or combined with those measured from the impedance system described herein, can indicate perfusion of vessels in the underlying tissue, as well as the concentration of blood cells within the vessel.

FIGS. 10A and 10B show, respectively, an image and photograph of the circuit board 14 used to control the patch. The circuit board 14 shown in the Figure comprises a 4-layer fiberglass/metal structure that includes metal pads attached, for example soldered to, among other components, an analog-to-digital converter 68, accelerometer 75, operational amplifiers 71a-f, and power regulators 72a-b. More specifically, in embodiments operational amplifiers 71a-d comprise analog high and low-pass filters, and operational amplifiers 71e-f and power regulators 72a-b collectively regulate power levels for the various components in the circuit board 14. In embodiments, an accelerometer 75 measures motion of the circuit board 62 and, in doing this, any part of the patient’s body it is attached to. For example, signals from the accelerometer 75 to determine if a patient wearing the patch is moving; this may indicate the presence of motion-related artifacts in time-dependent waveforms, such as that shown in FIG. 9. The analog-to-digital converter 68 digitizes analog impedance and optical waveforms after they have been filtered and converts them into digital waveforms with 16-bit resolution and a maximum digitization rate of 200 Ksamples/second (herein “Ksps”).

In embodiments, the circuit board 14 additionally includes sets of metal-plated holes that support a 4-pin connector 69, two 6-pin connectors 77, 78, and a 3-pin connector 79 that connect to external systems (e.g. LEDS, photodiodes). Through the connector 79 the circuit board receives power (+5V, +3.3V, and ground) from an external power supply, e.g. a battery located in the patch. These power levels may be different in other embodiments of the disclosure. In embodiments, digital signals and a corresponding ground from the analog-to-digital converter 68 are terminated at connector 78. The connector 77 is used primarily for testing and debugging purposes, and in particular allows analog impedance and optical signals, once they pass through analog high and low-pass filters, to be measured with an external device such as, for example, an oscilloscope.

In embodiments, the circuit board 14 additionally comprises components for processing, storing, and transmitting data that are digitized by the analog-to-digital converter 68. For example, the circuit board 14 can include a microprocessor, microcontroller, or similar integrated circuit, and can additionally provide analog and digital circuitry for the patch. In embodiments, the microprocessor or microcontroller thereon can operate computer code to process optical and impedance waveforms to determine vital signs, e.g. HR, or other parameters such as heart rate variability (herein “HRV”), respiratory rate (herein “RR”), blood pressure (herein “BP”), pulse oximetry (herein “SpO₂”), skin temperature (herein “TEMP”), CO, SV, and other parameters related to the patient’s perfusion status. “Processing” by the microprocessor in this way, as used herein, means using computer code or a comparable approach to digitally filter (e.g. with a high-pass, low-pass, and/or band-pass filter), mathematically manipulate, and generally process and analyze the waveforms and parameters and constructs derived therefrom with algorithms known in the art. Examples of such algorithms include those described in the following issued patents, the contents of which are incorporated herein by reference in their entirety: “NECK-WORN PHYSIOLOGICAL MONITOR”, U.S. Pat. 11,123,020; “NECKLACE-SHAPED PHYSIOLOGICAL MONITOR”, U.S. Pat. 11,141,072; and “BODY-WORN SENSOR FOR CHARACTERIZING PATIENTS WITH HEART FAILURE”, U.S. Pat. 11,129,537.

In related embodiments, the circuit board can comprise both flash memory and random-access memory for storing time-dependent waveforms and numerical values, either before or after processing by the microprocessor. In still other embodiments, the circuit board can comprise BLUETOOTH^{®} and/or Wi-Fi transceivers for both transmitting and receiving information to remote systems, e.g. wireless gateways, mobile phones, tablet computers, and traditional computers.

FIG. 11 illustrates an analog circuit 134 included in the circuit board shown in FIG. 10 that performs impedance measurement according to the disclosure. The Figure shows just one possible embodiment of the circuit 134; similar monitoring results can be achieved in further embodiments using a design and collection of electrical components that differ from those shown in the Figure.

The circuit 134 comprises a first current-injecting electrode region 138a that injects a high-frequency, low-amperage current (11) into a tissue area such as a skin flap associated with the patient. This serves as the current source. In embodiments, a current pump 140 provides the modulated current, with the modulation frequency typically being between 5-500 KHz and the mean current magnitude being between 0.1 mA and 1 mA. Preferably, the current pump 140 provides current that is modulated at 70 kHz through the first current-injecting electrode region 138a. In embodiments, a second current-injecting electrode region 138b also injects a high-frequency, low-amperage current (I2) into, for example, the patient’s thoracic cavity, with the current injected by the second current-injecting region 138b being 90° out of phase with respect to the current (I1) injected by the first current-injecting electrode region.

In embodiments, sensing-electrode regions 142a and 142b sense the time-dependent voltages encountered by the propagating current I1 and I2, respectively. These sensing-electrode regions are indicated in the figure as S1 and S2. Per Ohm’s law, the voltage sensed by these sensing-electrode regions 142a, 142b divided by the magnitude of the injected current yields a time-dependent resistance (i.e., impedance) that relates to blood flow in the underlying vessels. As shown by the waveform 144 in the Figure, the time-dependent resistance features a slowly varying DC offset, typically characterized by a parameter called “Z0”, that indicates the baseline impedance encountered by the injected current; for a typical impedance measurement, this will depend, for example, on the amount of fluids in the tissue area such as a skin flap, along with the fat, bone, muscle, and blood volume in the area. Z0, which typically has a value between about 10 and about 150 Ω, is also influenced by low-frequency, time-dependent processes such as respiration, particularly when the patch is worn on the patient’s chest. Such processes affect the inherent capacitance near the chest region that impedance measures and are manifested in the waveform by low-frequency undulations, such as those shown in the waveform 144. A relatively small (typically about 0.1-0.5 Ω) AC component, (herein “Z(t)”) lies on top of Z0 and is attributed to changes in resistance caused by the heartbeat-induced blood that propagates in the underlying vessels, as described in detail above. Z(t) is processed with a high-pass filter to form an impedance signal that features a collection of individual pulses 146 that are ultimately processed to determine perfusion in the underlying vessels, as described above.

In embodiments, voltage signals measured by the first sensing-electrode region 142a (S1) and the second sensing-electrode region 142b (S2) feed into a differential amplifier 148 to form a single, differential voltage signal which is modulated according to the modulation frequency (e.g., 70 kHz) of the current pump 140. In embodiments, the signal then flows to a demodulator 150, which also receives a carrier frequency from the current pump 140 to selectively extract signal components that only correspond to the impedance measurement. The collective function of the differential amplifier 148 and the demodulator 150 can be accomplished using many different circuits designed to extract weak signals-such as the AC impedance signal-from noise. For example, these components can be combined to form something equivalent to a “lock-in amplifier” that selectively amplifies signal components occurring at a well-defined carrier frequency. Or the signal and carrier frequencies can be deconvoluted in much the same manner as that used in a conventional AM radio using a circuit featuring one or more diodes.

The phase of the demodulated signal may also be adjusted with a phase-adjusting component 152 during the amplification process. In embodiments of a sensor according to the disclosure, the MAX30009 chipset, described above, can be used for this application. The latter measurement is performed with components for digital differential amplification, demodulation, and phase adjustment (such as those used for the impedance measurement) that are, in embodiments, integrated directly into the chipset.

In embodiments, once the impedance signal is extracted, it flows to a series of analog filters 154, 156, 158 within the circuit 134 that remove extraneous noise from the Z0 and Z(t) signals. The first low-pass filter 154 (30 Hz) removes any high-frequency noise components (e.g. power line components at 60 Hz) that may corrupt the signal. Part of the signal that passes through the filter 154, which represents Z0, is ported directly to a channel in an analog-to-digital converter 160. The remaining part of the signal feeds into a high-pass filter 156 (0.1 Hz), which passes high-frequency signal components responsible for the shape of individual pulses 146. This signal then passes through a final low-pass filter 158 (10 Hz) to further remove any high-frequency noise. Finally, the filtered signal passes through a programmable gain amplifier (PGA) 162, which, using a 1.65V reference, amplifies the resultant signal with a computer-controlled gain. The amplified signal represents Z(t) and is ported to a separate channel of the analog-to-digital converter 160, where it is digitized alongside of Z0. The analog-to-digital converter and PGA are integrated directly into the MAX30009 chipset described above. The chipset can simultaneously digitize waveforms such as Z0 and Z(t) with 24-bit resolution and sampling rates (e.g. 500 Hz) that are suitable for physiological waveforms. Thus, in theory, this one chipset can perform the function of the differential amplifier 148, demodulator 150, PGA 162, and analog-to-digital converter 160. Reliance on just a single chipset to perform these multiple functions ultimately reduces both size and power consumption of the impedance circuit 134.

In embodiments, the microprocessor 136 receives digitized Z0 and Z(t) signals through a conventional digital serial interface such as an SPI or I2C interface. Algorithms for converting the waveforms into actual measurements of perfusion are performed by the microprocessor 136. The microprocessor 136 also receives digital motion-related waveforms from the on-board accelerometer and processes these waveforms to determine parameters such as the degree/magnitude of motion, frequency of motion, posture, and activity level.

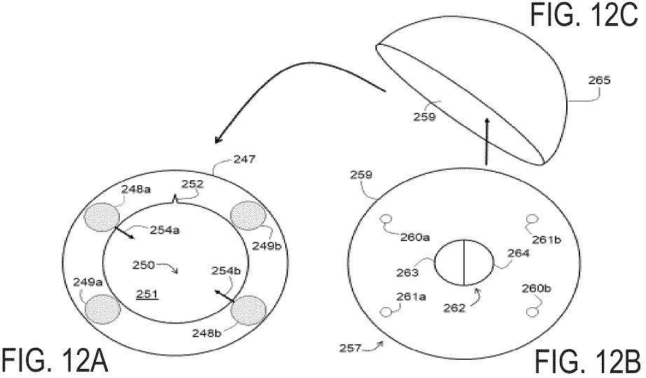

FIGS. 12A-C shows another embodiment of the disclosure featuring a single electrode patch 247 for measuring tissue perfusion using an impedance sensor similar to that described above. The electrode patch 247 includes four distinct electrodes: sense electrodes 248a,b and drive electrodes 249a,b. The electrode patch 247 is shaped as an annular ring with an opening 250 in its center. The annular ring’s bottom surface supports both the sense 248a,b and drive 249a,b electrodes surrounding the opening 250. The electrode patch 247 is typically composed of a stretchable material, similar, for example, to TEGADERM^{®} manufactured by 3 M (https://www.3m.eom/3M/en_US/p/c/b/tegaderm/i/health-care/medical/); the sense 248a,b and drive 249a,b electrodes are composed of, for example, a conductive wet or dry hydrogel integrated into the stretchable material. During a measurement, the electrode patch 247 connects to a bottom surface 259 of a circular, battery-powered circuit board 257 that forms the primary component of a device 265 that measures blood flow in underlying tissue. The circuit board 257 features a first set of electrical contacts 260a,b mated to the sense electrodes 248a,b, and a second set of electrical contacts 261a,b mated to the drive electrodes 249a,b. The electrical contacts 260a,b are typically thin metal films that, during use, electrically connect to the hydrogel electrodes 248a,b, 249a,b. In embodiments, during a measurement, and as described in detail above, the contacts 261a,b transmit alternating current into the patient’s skin through the drive electrodes 249a,b while the contacts 260a,b port corresponding bioelectric signals to ECG and impedance circuits on the circuit board 257.

In embodiments, discrete circuits on the circuit board 257 process the bioelectric signals (e.g., filter and amplify them) to generate, respectively, analog time-dependent ECG, impedance, and reactance waveforms. These are then digitized with an analog-to-digital converter to yield digital waveforms suitable for follow-on processing. A microprocessor operating on the circuit board 257 runs computer code that uses algorithms to process these types of waveforms, extract any relevant features (e.g., signal levels of the impedance and reactance waveforms, components from the ECG waveforms), and then processes these to estimate blood flow in the underlying tissue, and other physiological conditions (e.g. HR, HRV, RR) in the patient that might indicate perfusion and other disease states (e.g. arrhythmias).

Additionally, in embodiments the circuit board 257 comprises an optical sensor 262 that, in embodiments, features a collection of light sources 263 and a photodetectors 264 operating in a reflection-mode geometry to make a NIRS measurement of the underlying tissue, as described above. In embodiments, for example, the collection of light sources 263 may be an array of light sources (typically LEDs or laser diodes), each emitting radiation at a different wavelength. Alternatively, the collection of light sources 263 may be a “white” light source (e.g., a multi-wavelength LED or tungsten lamp) that emits a collection of wavelengths throughout the visible, infrared and ultraviolet spectra. The collection of photodetectors 264 typically comprises a single photodiode or an array of photodiodes. Alternatively, it can be an imaging system configured to collect a spatial image of the underlying skin. For example, in embodiments, a CCD camera can be configured to collect a spatial image of the underlying skin.

In embodiments, the electrode patch 247 is composed of a foam substrate with an adhesive layer on its bottom surface. The sense 248a,b and drive 249a,b electrodes are typically made from a hydrogel material that is typically adhesive, electrically conductive and features an electrical impedance matched to the patient’s skin. Electrical traces and contacts are typically composed of conductive materials, such as thin metal films or conductive ink. In embodiments, electrodes may also be dry electrodes made of metals (e.g., tin, silver, sintered Ag/AgCl, gold, platinum, and stainless steel) or polymers (e.g. rubber with additives).

FIG. 13 indicates how a patient 10 wearing a patch 12 that measures blood flow can be remotely monitored, e.g. by a clinician in a hospital. Such embodiments would be used, for example, to characterize a post-surgical patient recovering at home. During use, the patch 10 measures blood flow from the patient as described herein, and transmits information (indicated by the arrow 100) indicating the degree of perfusion in the patient 12 to a wireless gateway device 102. Transmission is typically done with a wireless system based on BLUETOOTH^{®} or Wi-Fi. The wireless gateway device 102 typically operates a downloadable software application that manages wireless communication with the patch 12. After receiving information from the patch 12, the wireless gateway device 102 transmits it (indicated by the arrow 106) through an internal cellular network or Wi-Fi network to a cloud-based system 108. The cloud-based system 108 is typically a software system operating in a remote data center. In embodiments, the cloud-based system 108 is part of a hospital data-management system (or another third-party software system) and can communicate with other software systems through a software interface, such as a Web Services interface. In embodiments, information from the cloud-based system 108 is transmitted (indicated by the arrow 106) to a data-analytics system 112, where software algorithms analyze the patch-generated information from the patient to better estimate blood flow in the region of interest. In embodiments, for example, the data-analytics system 112 operates algorithms based on conventional “decision-tree” logic to determine perfusion in the region of interest in the patient. Alternatively, the data-analytics system 112 may operate algorithms based on machine learning or artificial intelligence to estimate the degree of perfusion. Such algorithms may be particularly useful when the patch 12 includes multiple sensing modalities, e.g. both optical and impedance sensors as described here.

Disclosed embodiments can further comprise a mechanical housing. For example, in embodiments the mechanical housing can be worn on the patient’s body and enclose at least partially at least one of the control module, processor, and transmitter.

Still other embodiments of the disclosure are within the scope of the following claims.

### Commercial Products / Kits

The present systems and devices can be finished as a commercial product by the usual steps performed in the present field, for example by appropriate sterilization and packaging steps. The material according to the present disclosure can be finally sterile-wrapped so as to retain sterility until use and packaged (e.g. by the addition of specific product information leaflets) into suitable containers (boxes, etc.).

According to further embodiments, the disclosed systems and devices can also be provided in kit form combined with other components necessary for the desired use.

### Methods of Use

Methods of use of disclosed embodiments can comprise affixing a disclosed system or device to a patient. Disclosed embodiments can further comprise monitoring data recorded by the system or device, as well as recording the data for future analysis. For example, in embodiments, a disclosed sensor is reversibly affixed to an area of tissue to determine the degree of blood perfusion in the area. For example, following breast reconstruction surgery, a disclosed sensor is reversibly affixed to a skin flap.

Further embodiments comprise monitoring of blood perfusion over time to identify changes in a subject’s cardiovascular health. For example, in an embodiment, a patient reversibly attaches a disclosed sensor on a monthly basis to a predetermined area of tissue such as the leg, to gauge changes in tissue perfusion levels over time.

### EXAMPLES

The following non-limiting Examples are provided for illustrative purposes only in order to facilitate a more complete understanding of representative embodiments. This example should not be construed to limit any of the embodiments described in the present Specification.

### Example 1

### Monitoring of Post-Surgical Blood Flow

A disclosed wearable blood flow system is affixed to the “skin flap” tissue area of a patient who has undergone breast reconstruction surgery. The system provides real-time blood flow data which indicates the tissue is adequately perfused.

### Example 2

### Monitoring of Post-Surgical Blood Flow

A disclosed wearable blood flow system is affixed to a “skin flap” tissue area of a patient after placement of the flap to treat a large wound. The system provides real-time blood flow data which indicates the tissue is adequately perfused.

### Example 3

### Monitoring of Post-Surgical Blood Flow

A disclosed wearable blood flow system is affixed to a “skin flap” tissue area of a patient after placement of the flap to treat a large burn. The system provides real-time blood flow data which indicates the tissue is adequately perfused. Data collected by the system is transmitted via WiFi and a web services interface to a doctor who evaluates the perfusion in the treatment area.

### Example 4

### Monitoring of Post-Surgical Blood Flow

A disclosed wearable blood flow system is affixed to a “skin flap” tissue area of a patient after placement of the flap to treat an amputation wound. The system provides real-time blood flow data which indicates the tissue is adequately perfused. Data collected by the system is transmitted via BLUETOOTH^{®} and a web services interface to a doctor who evaluates the perfusion in the treatment area.

### Example 5

### Monitoring of Post-Surgical Blood Flow

A disclosed wearable blood flow system is affixed to a “skin flap” tissue area of a patient after placement of the flap to treat an amputation wound. The system is worn for the duration of their hospital stay and provides real-time blood flow data which indicates the tissue is adequately perfused. Data collected by the system is transmitted via WiFi and a web services interface to a doctor who evaluates the perfusion in the treatment area.

### Example 6

### Monitoring of Post-Surgical Blood Flow

A disclosed wearable blood flow system is affixed to the “skin flap” tissue area of a patient who has undergone breast reconstruction surgery and remains in place for 7 days. The system provides real-time blood flow data which indicates the tissue is adequately perfused. Data collected by the system is transmitted via WiFi and a web services interface to a doctor who evaluates the perfusion in the treatment area.

### Example 7

### Monitoring of Post-Surgical Blood Flow

A disclosed wearable blood flow system is affixed to a “skin flap” tissue area following surgery. The system is worn intermittently at a scheduled time of day for 10 days and provides real-time blood flow data which indicates the tissue is adequately perfused. Data collected by the system is transmitted via WiFi and a web services interface to a doctor who evaluates the perfusion in the treatment area.

### Example 8

### Monitoring of Post-Surgical Blood Flow

A disclosed wearable blood flow system is affixed to a “skin flap” tissue area following surgery. The system is worn intermittently at a scheduled time of day for 30 days and provides real-time blood flow data which indicates the tissue is adequately perfused. Data collected by the system is transmitted via BLUETOOTH^{®} and a web services interface to a doctor who evaluates the perfusion in the treatment area.

### Example 9

### Monitoring of Blood Flow

A disclosed wearable blood flow system is affixed to a tissue area of a patient where perfusion data is to be collected. The system provides real-time blood flow data which indicates the tissue is adequately perfused. Data collected by the system is transmitted via WiFi and a web services interface to a doctor who evaluates the perfusion in the treatment area.

### Example 10

### Monitoring of Blood Flow

A disclosed wearable blood flow system is affixed to a tissue area of a patient where perfusion data is to be collected intermittently over 30 days. The system provides real-time blood flow data which indicates the tissue is adequately perfused. Data collected by the system is transmitted via WiFi and a web services interface to a doctor who evaluates the perfusion in the treatment area.

### Example 11

### Monitoring of Blood Flow

A disclosed wearable blood flow system is affixed to a tissue area of a patient where perfusion data is to be collected intermittently over 60 days. The system provides real-time blood flow data which indicates the tissue is adequately perfused. Data collected by the system is transmitted via WiFi and a web services interface to a doctor who evaluates the perfusion in the treatment area.

### Disclosed Embodiments

Embodiment 1. A system for characterizing blood flow in a blood vessel comprising:
an impedance system comprising at least one current-injecting electrode configured to inject an electrical current into the vessel, and at least one signal-measuring electrode configured to measure an impedance signal affected by the injected electrical current and blood flow in the vessel, wherein the current-injecting and signal-measuring electrodes are connected directly to the vessel; and
a processing system configured to receive the impedance signal from the impedance system, or a signal determined therefrom, and then process it to determine a parameter related to the blood flow in the vessel.

Embodiment 2. The system of embodiment 1, wherein at least one of the current-injecting and signal-measuring electrodes comprises a conductive material.

Embodiment 3. The system of embodiment 2, wherein at least one of the current-injecting and signal-measuring electrodes comprises a resorbable material.

Embodiment 4. The system of embodiment 3, wherein at least one of the current-injecting and signal-measuring electrodes comprises a material selected from the group consisting of a conductive polymer, zinc, iron, magnesium, and manganese.

Embodiment 5. The system of embodiment 1, wherein the impedance system further comprises a body-worn patch that connects to the at least one of the current-injecting and signal-measuring electrodes.

Embodiment 6. The system of embodiment 5, wherein the body-worn patch comprises a circuit board comprising an impedance circuit.

Embodiment 7. The system of embodiment 6, wherein the circuit board connects through a cable to the at least one of the current-injecting and signal-measuring electrodes.

Embodiment 8. The system of embodiment 7, wherein the cable comprises a conductive material.

Embodiment 9. The system of embodiment 8, wherein the cable comprises a resorbable material.

Embodiment 10. The system of embodiment 9, wherein the cable comprises a material selected from the group consisting of a conductive polymer, zinc, iron, magnesium, and manganese.

Embodiment 11. The system of embodiment 1, wherein the impedance signal is a time-domain waveform.

Embodiment 12. The system of embodiment 11, wherein the processing system is further configured to process the time-domain waveform to measure one or more heartbeat-induced pulses.

Embodiment 13. The system of embodiment 12, wherein the processing system is further configured to process the heartbeat-induced pulses to determine the parameter related to blood flow in the vessel.

Embodiment 14. The system of embodiment 1, wherein the impedance system is further configured to measure a capacitance value of the vessel.

Embodiment 15. The system of embodiment 14, wherein the processing system is further configured to process the capacitance value to determine the parameter related to blood flow in the vessel.

Embodiment 16. The system of embodiment 15, wherein the capacitance value is a resonant frequency corresponding to the vessel.

Embodiment 17. The system of embodiment 1, wherein the impedance system is configured to inject electrical current into the vessel at a single frequency.

Embodiment 18. The system of embodiment 1, wherein the impedance system is configured to inject electrical current into the vessel at multiple, unique frequencies.

Embodiment 19. The system of embodiment 1, further comprising an optical system configured to measure optical signals from the vessel.

Embodiment 20. The system of embodiment 19, wherein the optical system comprises at least one light source and at least one photodetector.

Embodiment 21. The system of embodiment 20, wherein the at least one light source is configured to emit optical radiation in the infrared spectral region.

Embodiment 22. The system of embodiment 21, wherein the optical system is further configured to measure an optical spectrum in an infrared spectral region.

Embodiment 23. The system of embodiment 1, further comprising an accelerometer.

Embodiment 24. The system of embodiment 23, wherein the processing system is further configured to measure motion-related signals from the accelerometer to determine motion corresponding to the patient.

Embodiment 25. The system of embodiment 1, further comprising a wireless transmitter.

Embodiment 26. The system of embodiment 25, wherein the wireless transmitter is further configured to transmit the parameter related to the blood flow in the vessel, or a parameter derived therefrom, to a remote system.

Embodiment 27. The system of embodiment 26, wherein the remote system is selected from the group consisting of a computer, mobile telephone, tablet computer, server, and cloud-based system.

Embodiment 28. The system of embodiment 25, wherein the wireless transmitter is selected from the group consisting of transmitters operating on BLUETOOTH^{®}, Wi-Fi, and cellular protocols.

Embodiment 29. A system for characterizing blood flow in a blood vessel, comprising:
an electrical system comprising at least one current-injecting electrode configured to inject an electrical current into the vessel, and at least one signal-measuring electrode configured to measure an electrical signal affected by the injected electrical current and blood flow in the vessel, wherein the current-injecting and signal-measuring electrodes are connected directly to the vessel; and
a processing system configured to receive the electrical signal from the electrical system, or a signal determined therefrom, and then process it to determine a parameter related to the blood flow in the vessel.

Embodiment 30. A system for characterizing blood flow in a blood vessel, comprising:
an impedance system comprising at least one current-injecting electrode configured to inject an electrical current into the vessel, and at least one signal-measuring electrode configured to measure an impedance signal affected by the injected electrical current and blood flow in the vessel, wherein the current-injecting and signal-measuring electrodes are connected directly to the vessel;
an optical system comprising a light source and a photodetector, with the light source configured to irradiate the vessel with optical radiation and the photodetector configured to detect the optical radiation after it irradiates the vessel and generate an optical signal; and
a processing system configured to receive the impedance signal from the impedance system and the optical signal from the optical system, or a signals determined therefrom, and collectively process them to determine a parameter related to the blood flow in the vessel.

Embodiment 31. A system for characterizing blood flow in a blood vessel, comprising:
a body-worn patch comprising an impedance circuit;
an electrode system attached directly to the vessel and electrically connected to the impedance circuit; and
a processing system configured to receive signals from the impedance circuit and process them to estimate blood flow in the vessel.

Embodiment 32. The system of any of embodiments 1-31, wherein said blood vessel comprises a vein or artery.

Embodiment 33. A method for characterizing blood flow in a blood vessel, comprising applying the system of any of embodiments 1-32 to an area where blood flow is to be measured.

In closing, it is to be understood that although aspects of the present Specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to, or alternative configurations of, the disclosed subject matter can be made in accordance with the teachings herein without departing from the spirit of the present Specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the Specification is deemed to contain the group as modified thus fulfilling the written description of all Markush groups used in the appended claims.

Unless otherwise indicated, all numbers expressing a characteristic, item, quantity, parameter, property, term, and so forth used in the present Specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the Specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present Specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present Specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A system for characterizing blood flow in a blood vessel comprising:
an impedance system comprising at least one current-injecting electrode configured to inject an electrical current into the vessel, and at least one signal-measuring electrode configured to measure an impedance signal affected by the injected electrical current and blood flow in the vessel, wherein the current-injecting and signal-measuring electrodes are connected directly to the vessel; and
a processing system configured to receive the impedance signal from the impedance system, or a signal determined therefrom, and then process it to determine a parameter related to the blood flow in the vessel.

2. The system of claim 1, wherein at least one of the current-injecting and signal-measuring electrodes comprises a conductive material.

3. The system of claim 2, wherein at least one of the current-injecting and signal-measuring electrodes comprises a resorbable material.

4. The system of claim 3, wherein at least one of the current-injecting and signal-measuring electrodes comprises a material selected from the group consisting of a conductive polymer, zinc, iron, magnesium, and manganese.

5. The system of claim 1, wherein the impedance system further comprises a body-worn patch that connects to the at least one of the current-injecting and signal-measuring electrodes.

6. The system of claim 5, wherein the body-worn patch comprises a circuit board comprising an impedance circuit.

7. The system of claim 6, wherein the circuit board connects through a cable to the at least one of the current-injecting and signal-measuring electrodes.

8. The system of claim 7, wherein the cable comprises a conductive material.

9. The system of claim 8, wherein the cable comprises a resorbable material.

10. The system of claim 9, wherein the cable comprises a material selected from the group consisting of a conductive polymer, zinc, iron, magnesium, and manganese.

11. The system of claim 1, wherein the impedance signal is a time-domain waveform.

12. The system of claim 11, wherein the processing system is further configured to process the time-domain waveform to measure one or more heartbeat-induced pulses.

13. The system of claim 12, wherein the processing system is further configured to process the heartbeat-induced pulses to determine the parameter related to blood flow in the vessel.

14. The system of claim 1, wherein the impedance system is further configured to measure a capacitance value of the vessel.

15. The system of claim 14, wherein the processing system is further configured to process the capacitance value to determine the parameter related to blood flow in the vessel.

16. The system of claim 15, wherein the capacitance value is a resonant frequency corresponding to the vessel.

17. The system of claim 1, wherein the impedance system is configured to inject electrical current into the vessel at a single frequency.

18. The system of claim 1, wherein the impedance system is configured to inject electrical current into the vessel at multiple, unique frequencies.

19. The system of claim 1, further comprising an optical system configured to measure optical signals from the vessel.

20. The system of claim 19, wherein the optical system comprises at least one light source and at least one photodetector.

21. The system of claim 20, wherein the at least one light source is configured to emit optical radiation in the infrared spectral region.

22. The system of claim 21, wherein the optical system is further configured to measure an optical spectrum in an infrared spectral region.

23. The system of claim 1, further comprising an accelerometer.

24. The system of claim 23, wherein the processing system is further configured to measure motion-related signals from the accelerometer to determine motion corresponding to the patient.

25. The system of claim 1, further comprising a wireless transmitter.

26. The system of claim 25, wherein the wireless transmitter is further configured to transmit the parameter related to the blood flow in the vessel, or a parameter derived therefrom, to a remote system.

27. The system of claim 26, wherein the remote system is selected from the group consisting of a computer, mobile telephone, tablet computer, server, and cloud-based system.

28. The system of claim 25, wherein the wireless transmitter is selected from the group consisting of transmitters operating on BLUETOOTH^{®}, Wi-Fi, and cellular protocols.

29. A system for characterizing blood flow in a blood vessel, comprising:
an electrical system comprising at least one current-injecting electrode configured to inject an electrical current into the vessel, and at least one signal-measuring electrode configured to measure an electrical signal affected by the injected electrical current and blood flow in the vessel, wherein the current-injecting and signal-measuring electrodes are connected directly to the vessel; and
a processing system configured to receive the electrical signal from the electrical system, or a signal determined therefrom, and then process it to determine a parameter related to the blood flow in the vessel.

30. A system for characterizing blood flow in a blood vessel, comprising:
an impedance system comprising at least one current-injecting electrode configured to inject an electrical current into the vessel, and at least one signal-measuring electrode configured to measure an impedance signal affected by the injected electrical current and blood flow in the vessel, wherein the current-injecting and signal-measuring electrodes are connected directly to the vessel;
an optical system comprising a light source and a photodetector, with the light source configured to irradiate the vessel with optical radiation and the photodetector configured to detect the optical radiation after it irradiates the vessel and generate an optical signal; and
a processing system configured to receive the impedance signal from the impedance system and the optical signal from the optical system, or a signals determined therefrom, and collectively process them to determine a parameter related to the blood flow in the vessel.

31. A system for characterizing blood flow in a blood vessel, comprising:
a body-worn patch comprising an impedance circuit;
an electrode system attached directly to the vessel and electrically connected to the impedance circuit; and
a processing system configured to receive signals from the impedance circuit and process them to estimate blood flow in the vessel.

32. The system of any of claims 1-31, wherein said blood vessel comprises a vein or artery.

33. A method for characterizing blood flow in a blood vessel, comprising applying the system of any of claims 1-32 to an area where blood flow is to be measured.
